(19) United States
(12) Patent Application Publication
(10) Pub. No.: US 2018/0342313 A1
(43) Pub. Date: Nov. 29, 2018

(54) SMART SUGGESTER SYSTEM

(71) Applicant: Praxify Technologies, Inc., Palo Alto, CA (US)

(72) Inventors: Abhijit Gupta, Pune (IN); Mohan Rao, Pune (IN)

(21) Appl. No.: 15/991,396

(22) Filed: May 29, 2018

(30) Foreign Application Priority Data
May 29, 2017 (IN) 201721018839

(51) Int. Cl. G16H 20/00; G16H 80/00; G16H 50/20; G16H 10/60

(52) U.S. Cl. CPC G16H 20/00; G16H 10/60; G16H 50/20; G16H 80/00

(57) ABSTRACT

Methods, systems, and apparatus, including computer programs encoded on computer storage media for a smart suggester system. Implementations can include receiving data indicating a chief complaint relating to a patient during a doctor-patient interaction. The system receives data indicating vitals relating to the patient. The system receives data indicating symptoms relating to the patient. The system generates a treatment plan based on data indicating the chief complaint, data indicating the vitals, and data indicating the symptoms related to the patient. The system provides the treatment plan to a client device of a doctor.

### CROSS-REFERENCE TO RELATED APPLICATIONS

This application claims priority under 35 U.S.C. § 119 to Indian Application No. 201721018839, filed on May 29, 2017, which is hereby incorporated by reference in its entirety.

### TECHNICAL FIELD

This specification generally relates to the field of information systems, computational systems, databases, and networking systems, and communication systems.

### BACKGROUND

Medical practice entails activities in relation to health and body, surgical procedures, examination procedures, diagnostic procedures, prognosis procedures, and the like activities. Qualified medical professional are equipped to deal with various facets of medical practice; in relation to the academic qualification that they have reached, in relation to the professional experience that they have gained.

### SUMMARY

One general aspect of this subject matter relates to the field of healthcare information, healthcare technology, healthcare management, practice management, electronic medical records, and electronic health records. Additionally, the subject matter relates to a smart suggester system and method

The terms medical record, health record, encounter and medical chart are used somewhat interchangeably to describe the systematic review and documentation of a single patient's medical or health journey that include a patient's history, diagnosis, prognosis, symptoms, vitals, review of systems, physical examination, medications, lab and diagnostics, allergies, surgical procedures and care across time not just within one particular health care provider setting, but also covering multiple health care providers and their interactions with the patient in context.

Medical records include a variety of notes and data relating to doctor-patient interaction, doctor's interpretation of patient's complaints, diagnosis, prognosis, investigations and treatment plans. This data can include signs and symptoms data, review of various body systems data, examination data, vitals data, diagnosis data, medical decision making data, medical history data, family history, social history, previous surgical procedures and hospitalizations, any specific historical data of medicines taken, allergies, chronic and acute problems, lab reports, radiology images and reports' data, other investigation results' data, input/output data, drugs and immunization administration data and medication data, prognosis data, visit notes, insurance data, demographics, other relevant health histories, genomic data, data from wearables and other medical devices, and the like. Reviewing and maintenance of complete and accurate medical records is essential for the doctor as well as the patient for ensuing accurate diagnosis and treatment also from a general health and wellness perspective as well from a legal perspective.

Medical records are used to understand the patient's current health status and past health history to ensure patient wellness and also to identify patient's diagnosis and provide/recommend relevant treatment protocol to a patient or fellow care providers for treating patients. Medical records can also be used as an aid to supplement the judgement and decision of a doctor/care provider. This system includes data of a patient that is captured at various stages of his/her life and is used for a variety of medical and analytical purposes.

The types of personal health information that can be included in the medical records may cover the following: patient demographics information including, but not limited to, name, gender, birth date, blood type, race, ethnicity, marital status, address/geographical location, emergency contact information; a complete history of patients past visit histories; date of last physical exam; dates and results of tests and screenings; major illnesses and surgeries, with dates; a list of medicines, dosages and how long they are being taken; any allergies and its reactions; any chronic/acute diseases and treatment plans; any history of illnesses in your family; dates and results of lab tests, imaging tests, and screenings; social history, family history; immunizations; risk assessments; care plans; vitals; data from wearables; genomic data; and, various clinical assessments and scores.

A care management system typically includes comprehensive medical records of patients and a set of procedures and protocols that a doctor prescribes for a patient. In its electronic format, patient centered electronic medical record systems involve all the aspects of patient and illness/disease management, steps pertaining to which are described above and may generally be referred during a patient-doctor interaction or for treating patients or for evolving better treatment protocols for future patients. For a doctor to review and record all aspects or facets of a patient, during the doctor-patient interaction, the patient centered electronic medical record systems must be intuitive towards the workflows of that particular doctor and keep in context the various aspects of patient's demographic and medical information. Thus, these systems must provide patient medical information views to clinicians such that they spend as little time as possible to find relevant information to ensure better diagnosis and medical decision making for treatment protocols and document important facets of the patient's encounter, and focus more on patient care as much as possible. Intuitiveness in this case means the ability of the system to understand how a clinician practices, learn from how the clinician practices, and be able to provide the right workflow, so that the clinician does not waste time in searching for information relevant in the context of the patient and his/her history and for documenting a patient record.

Typically, a patient is profiled in terms of demographics, medical history, family history, social history, current context (relating to season, epidemic, travel history, and the like), previous surgeries' history, investigations, vitals, current and previous problems, allergies, immunizations, and the like.

In some implementations, a ‘doctor-patient interaction’ is meant to include the steps from understanding patient reported complaints, reviewing patient's medical records in the context of the complaints and condition, documenting history of present illnesses, to reviewing of body systems, to doing physical examination of the patient, to diagnosis to treatment plan to prognosis. In this context, the system can be used in correlation with this doctor-patient interaction is ready to ‘understand’ the interaction.

In some implementations, the system can be context-aware so that the system understands the doctor correctly in terms of pre-defined parameters.

In some implementations, the system can be context-aware so that the system understands the patient correctly in terms of pre-defined parameters.

In some implementations, the system can be context-aware so that the system understands the patient's previous records and histories correctly in terms of pre-defined parameters.

In some implementations, the system can be context-aware so that the system understands the doctor-patient interaction correctly in terms of pre-defined parameters.

In some implementations, the system can be context-aware so that the system understands the location and seasons correctly in terms of pre-defined parameters.

In some implementations, the system can be context-aware so that the system understands the demographic correctly in terms of pre-defined parameters.

In some implementations, the system can be context-aware so that the system understands how the doctor is interacting with the patient, how the doctor reviews medical records, and how the doctor documents in the patient centered electronic medical record system in terms of pre-defined parameters.

In some implementations, the system can be context-aware so that the system understands the current condition or state of the patient while documenting in the patient centered electronic medical record system in terms of pre-defined parameters.

In some implementations, the system can be context-aware so that the system understands which data set, for example, vitals and lab results, of the medical record should be promoted of the patient while reviewing patient medical records and documenting in the patient centered electronic medical record system in terms of pre-defined parameters.

In some implementations, the system can be context-aware to understand which actions, for example, adding a particular problem or suggesting a particular test, of the medical record should be promoted of the patient while documenting in the patient centered electronic medical record system in terms of pre-defined parameters.

The intelligent and intuitive system and method can be configured and designed so that a doctor is enabled and empowered to interact with the system in a context-aware manner. Therefore, the system and method context-aware and context-ready can be utilized so a doctor can interact with it.

With the advent of Internet of Things (IOT) and mobility, wearable devices and sensors have become ubiquitous in nature. Doctors today face a huge problem on understanding contexts from the trillions of bytes of information that they get from their patients. The vastness of this data needs to be interpreted by intelligent systems, and has to be presented to a doctor in a manner which will make logical sense for decision making. This intelligent system needs to be aware of various contexts in which these datasets were captured by these devices. Those contexts need to be interpreted in real time to aid the doctor to not take unnecessary interventions or measures, which will increase healthcare costs.

Also, each doctor has his/her own way of practicing and consuming patient data. The data needs to communicate to the doctor, what he/she is looking for answers to take real time decisions at point-of-care. This method of synthesizing data with various contexts and presenting the data to the doctor is important. Additionally, another specifically important premise is an application, which provides a method of suggesting context-relevant information to a doctor.

It is important that an intelligent and intuitive system and method be configured and designed to that a doctor is enabled and empowered to reach a correct treatment plan taking into cognizance the various factors that correlate with a patient as well as associated factors.

Therefore, based on a patient-doctor interaction, a treatment plan needs to be auto-formed and auto-configured. This auto-formation and auto-configuration, typically, over time, iteratively, relegates the need to type and input data, thereby providing a relatively faster and a relatively more accurate mechanism for suggesting inputs.

In general, one innovative aspect of the subject matter disclosed described in this specification can be embodied in methods that include the actions of receiving, by the one or more processors, data indicating a chief complaint relating to a patient during a doctor-patient interaction; receiving, by the one or more processors, data indicating vitals relating to the patient; receiving, by the one or more processors, data indicating symptoms relating to the patient; generating, by the one or more processors, a treatment plan based on data indicating the chief complaint, data indicating the vitals, and data indicating the symptoms related to the patient; and providing, by the one or more processors, the treatment plan to a client device of a doctor.

Other innovative aspects of the subject matter disclosed described in this specification can be embodied in methods that include determining a location of the doctor corresponding to the doctor-patient interaction.

In some implementations, the method further includes determining a location of the patient corresponding to the doctor-patient interaction.

In some implementations, the method further includes determining a profile for the doctor, wherein the profile for the doctor includes a type of specialty, doctor treatment plan preferences, and doctor clinical pathways preferences.

In some implementations, the method further includes wherein the treatment plan comprises at least one of laboratories tests, tests, follow-ups, recommendations, allergies, and medications.

In some implementations, the method further includes further comprising: generating, by the one or more processors, weight factors based on the data indicating the chief complaint relating to the patient, the data indicating the vitals relating to the patient, and the data indicating the symptoms relating to the patient; and assigning, by the one or more processors, the weight factors to patient data items to assist with generating the treatment plan.

In some implementations, the method further includes wherein the patient data items comprise at least one of disease data items, treatment plan data items, treatment protocol data items, and clinical pathway data items.

Other embodiments of this aspect include corresponding computer systems, apparatus, and computer programs recorded on one or more computer storage devices, each configured to perform the actions of the methods. A system of one or more computers can be configured to perform particular operations or actions by virtue of software, firmware, hardware, or any combination thereof installed on the system that in operation may cause the system to perform the actions. One or more computer programs can be configured to perform particular operations or actions by virtue of including instructions that, when executed by data processing apparatus, cause the apparatus to perform the actions.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 illustrates an example of a smart suggester system.

Like reference numbers and designations in the various drawings indicate like elements.

### DETAILED DESCRIPTION

This specification seeks to provide a system and method for electronic medical and health records. The system and method for electronic medical and healthcare records, which provides for practice management. Additionally, this system seeks to improve health care quality.

This specification seeks to provide a system and method for recording at least a facet of a patient and doctor interaction, such as during a patient doctor visit.

An additional object of the specification is to provide a system and method for providing a touch based, click based, voice based or gesture based recording of at least a facet of patient-doctor interaction/visit.

Yet an additional object of the specification is to provide an intuitive system and method for recording a patient-doctor interaction/visit.

Still an additional object of the specification is to provide a treatment plan auto-configuration mechanism.

Another additional object of the specification is to provide a treatment plan auto-formation mechanism.

An additional object of the specification is to provide a system and method, which is easy to use and understand for doctors as well as for patients, thereby increasing user adaptability.

Yet an additional object of the specification is to provide a system and method, which provide a treatment plan in an accurate manner considering a patient's medical profile.

Still an additional object of the specification is to provide a system and method, which enable and empower a doctor to arrive at a correct treatment plan taking into cognizance the various factors that correlate with a patient as well as associated factors.

Another additional object of the specification is to provide a system and method, which enable and empower a doctor to record at a correct treatment plan taking into cognizance the various factors that correlate with a patient as well as associated factors.

Yet another additional object of the specification is to provide a system and method, which iteratively learns treatment plan auto-formation parameters.

Still another additional object of the specification is to provide a system and method, which iteratively learns treatment plan auto-configuration parameters.

Yet another additional object of the specification is to provide a system and method, which relegates the need to need to type and input data, typically relating to treatment plans, thereby providing a relatively faster and a relatively more accurate mechanism for forming a treatment plan.

For the purposes of this specification, the term, ‘doctor’ would include without limitations doctor, doctors, physicians, specialists, super specialists, dentists, surgeons, physiologists, psychiatrists, hospitalists, physiotherapists, medics, medical practitioners, medicos, nurses, nurse practitioners, physician assistants, paramedics, midwifes, clinical staff, and the likes of hospital related or healthcare related persons who deal with patients.

For the purposes of this specification, a ‘tap’ is defined as a touch or a haptic contact or haptic engagement (whether discrete or continuous) or a click or a gesture, in response to which a pre-defined task or action takes place.

For the purposes of this specification, the term, ‘care management’, is meant to include actions, set of procedure and protocols adhered in a healthcare environment, which may include, but are not limited to scheduling, patient registration, patient onboarding, patient related document management, patient account management, billing, claims' processing, illness management, diagnosis, prognosis, examination, tests, results, interconnecting various nodes in the healthcare ecosystem, notifications and alarms, and the like.

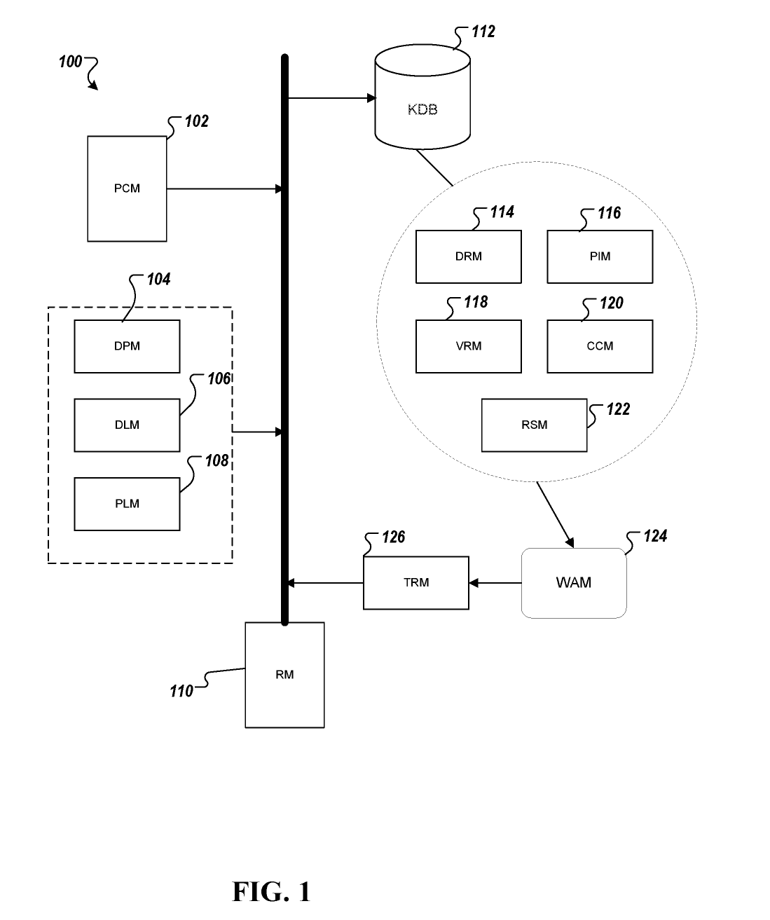

FIG. 1 illustrates an example of a smart suggester system 100. In some implementations, the system 100 includes a profile configuration mechanism (PCM) 102 adapted to define, configure, and render a patient profile. Each patient profile includes profile fields, which are to be populated with profile data items and parameters relating to these profile data items, hereinafter called profile data parameters. Each of the profile data items are tagged and weighted as per relevant context.

Typically, a patient's profile includes profile fields which relate to demographics, medical history, previous encounters, physicians, problems, diagnosis, allergies, vitals, signs, weights, measurements, growth chart, lines and tubes, intake and output measurements, immunizations and schedule, labs, microbiology, pathology, administered medications, home medications, notes (progress notes, nursing notes, other clinically relevant notes), outstanding orders, diagnostic results (reports, images, and the like), code status, respiratory treatment, family history, social history, previous surgical and/or hospitalization history, any other specialty specific history, risk scores, various assessments, current complaints, adverse reactions, current context (relating to season, epidemic, location, travel, genetics, race, ethnicity and the like), discharge summaries, visit summaries, genomic data of the patient, role of a user, department and specialty, care setting, and the like important event notifications.

Each of these profile items corresponds to a context, which is further used in this system and method in order to suggest a context-aware rendition of suggestion(s).

Each of these data input items are stored in a corresponding database. These databases may be correlated to each other or be standalone.

In some implementations, the system 100 includes a doctor profile defining mechanism (DPM) 104 configured to define and profile a doctor. Typically, a doctor's profile includes a type of specialty, doctor preferences in terms of treatment plan, doctor preferences in terms of clinical pathways, and the like.

In some implementations, the system 100 includes a doctor location determination mechanism (DLM) 106 configured to determine a doctor's location. This aids in factoring in local and hyperlocal conditions such as seasons, external conditions, and the like.

In some implementations, the system 100 includes a patient location determination mechanism (PLM) 108 configured to determine a patient's location. This aids in factoring in local and hyperlocal conditions such as seasons, external conditions, and the like.

In some implementations, the system 100 includes a rendering mechanism (RM) 110 configured to capture inputs in various templates in a step-wise manner. These steps relate to steps from capturing details to providing a treatment plan. These inputs relate to auto-formed and auto-suggested inputs learned by this system and method. These inputs also relate to patient inputs and doctor inputs. Typically, the rendering mechanism 110 is configured to work on at least these three types of inputs: a) profile of doctor; b) location; and c) chief complaint. In some implementations, the specification also defines a patient condition parameter.

The rendering mechanism 110 is also configured to make mapped suggestions. This is achieved by weight assignment, which is sought by determination of frequency of use, determination of heat map, and the like.

In some implementations, the system 100 includes a chief complaint registering mechanism (CCM) 120 configured to records at least a chief complaint relating to a patient during a doctor-patient interaction. This is called from an existing database of chief complaints. This chief complaint record acts as a first trigger towards forming or configuring a treatment plan correlating to a patient. The chief complaint is tagged with treatment protocols. In some implementations, the chief complaint is recorded by a touch gesture from an existing list of chief complaints. In some implementations, the chief complaint is recorded by a voice to text conversion mechanism.

In some implementations, the system 100 includes a history of present illness recording mechanism (PIM) 116 configured to record symptoms of a patient. Based on inputs from the rendering mechanism 110, fields in this recording mechanism 116 are shown. Further, based on inputs from the rendering mechanism 110, fields in this recording mechanism 116 are auto-suggested and/or auto-populated by means of an auto-suggestion mechanism trained for such purposes.

In some implementations, the system 100 includes a vitals recording mechanism (VRM) 118 configured to record vitals of a patient. Based on inputs from the rendering mechanism 110, fields in this recording mechanism 116 are shown. Further, based on inputs from the rendering mechanism 110, fields in this recording mechanism 116 are auto-suggested and/or auto-populated. Additionally, inputs from the history of present illness recording mechanism 116 may be used so that fields in this recording mechanism are auto-suggested and/or auto-populated.

In some implementations, the system 100 includes a review of systems recording mechanism (RSM) 122 configured to record data of a patient. Based on inputs from the rendering mechanism 110, fields in this recording mechanism 116 are shown. Further, based on inputs from the rendering mechanism 110, fields in this recording mechanism 116 are auto-suggested and/or auto-populated.

Additionally, inputs from the vitals recording mechanism 118 may be used so that fields in this recording mechanism 116 are auto-suggested and/or auto-populated by means of an auto-suggestion mechanism trained for such purposes.

In some implementations, the system includes an assessment/diagnosis recording mechanism (DRM) 114 configured to record assessment/diagnosis parameters and data items of a patient. Based on inputs from the rendering mechanism 110, fields in this recording mechanism 116 are shown. Further, based on inputs from the rendering mechanism 110, fields in this recording mechanism 116 are auto-suggested and/or auto-populated by means of an auto-suggestion mechanism trained for such purposes. Additionally, inputs from the review of systems recording mechanism (RSM) 122 may be used so that fields in this recording mechanism 116 are auto-suggested and/or auto-populated.

In some implementations, the system 100 includes a treatment plan rendering mechanism (TRM) 126 configured to render treatment plan data items for a patient. Data items of a treatment plan typically include laboratories, tests, follow-ups, recommendations, allergies, medications, and the like.

In some implementations, the system 100 includes a weight assignment mechanism (WAM) 124 configured to assign weight to inputs. Weight assignment is done based on factors such as patient profile data input, doctor profile data input, patient location data input, and the like. A mapping mechanism is configured to map assigned weighted items to a set of items including disease data items, treatment plan data items, treatment protocol data items, clinical pathway data items, and the like data items.

In order to suggest the auto-suggestion, relationships between various databases need to be intelligently formed. Furthermore, these relationships need to be context-aware and change dynamically in relation to real-time data.

Therefore, the system 100 includes a knowledge database (KDB) 112 and an electronic health record database—each of which include entities (parameters) correlated using this system and method. This correlation helps correlate data items with respect to these entities (parameters). In at least one embodiment, there is provided a suggestions' database including data items, which correlate with data structures. Furthermore, contextual tags may be defined in order to assign weights to data items. Rules are defined such that the weights define outputs in terms of suggestions i.e. data items from the suggestions' database. In at least one embodiment, the suggestion data items in the suggestions' database relates to treatment plan for a patient.

In some implementations, data items include inputs from journals, websites, case studies, textbooks, and the like literature. Initially, these data items are structured using pre-defined rules of structuring and association in relation with pre-defined structured matrices such as snomed. These rules are auto-learned intelligently, over time. Knowledge database 112 is built/influenced by: 1) context related findings; 2) evidence based protocols; 3) ontological mapping; 4) differential diagnosis mapping; 5) external reasons such as contexts (which include location, doctor profile, patient profile).

Knowledge database (KDB) 112 influences everything to provide mapped suggestions at multiple levels. Context determination is done and context is a bias provided to items in the knowledge database by tagging, indexing, referencing, cross-referencing, or the like mechanism.

In one example, the system detects high glucose levels over a defined period of time—checks system for HBA1C test—not done by the patient—so the system and method suggests a detection of the high glucose levels automatically.

This suggestion is a mapped suggestion provided by a rule engine, rules of which are set based on KDB and weights and context.

Weights are assigned by frequency and heat maps help define rules over a period of time.

In some implementations, the system includes an existing data feed of patient data. Data items in this database are also structured using pre-defined rules of structuring and association in relation with pre-defined structured matrices such as snomed. These rules are auto-learned intelligently, over time.

In some implementations, the electronic health record database is also communicably coupled to a real time feed of patient data. Real time feed of patient data includes data items which are contextually tagged. Contextual tagging may assign weights to data items. Contextual tagging allows for outputting correlative data items from the suggestions' database.

Since data items in both the databases, i.e. the knowledge database and the electronic health record database are similarly structured, the data items can be mapped, compared, displayed correlatively in order to identify patterns, form relationships, and provide suggestions relating to treatment plans.

In some implementations, one technical benefit includes providing a system and method, which iteratively provides or auto-forms or auto-configures a treatment plan for a given patient profile. This may be based on a chief complaint provided by the patient. Furthermore, charts are pre-empted in terms of their context, in terms of their fields, in terms of data in the fields—based on inputs and based on previous diagnosis or protocols as well as context. The technology in this specification focuses on how relationships between databases are formed, how real-time data is used in a context-aware manner in order to provide suggestions that provide a treatment plan of a patient.

The data, in each of the components, means, modules, mechanisms, units, devices of the system and method may be ‘encrypted’ and suitably ‘decrypted’ when required.

The systems described herein can be made accessible through a portal or an interface which is a part of, or may be connected to, an internal network or an external network, such as the Internet or any similar portal. The portals or interfaces are accessed by one or more of users through an electronic device, whereby the user may send and receive data to the portal or interface which gets stored in at least one memory device or at least one data storage device or at least one server, and utilizes at least one processing unit. The portal or interface in combination with one or more of memory device, data storage device, processing unit and serves, form an embedded computing setup, and may be used by, or used in, one or more of a non-transitory, computer readable medium. In at least one embodiment, the embedded computing setup and optionally one or more of a non-transitory, computer readable medium, in relation with, and in combination with the said portal or interface forms one of the systems of the technology. Typical examples of a portal or interface may be selected from but is not limited to a website, an executable software program or a software application.

The systems and methods may simultaneously involve more than one user or more than one data storage device or more than one host server or any combination thereof.

A user may provide user input through any suitable input device or input mechanism such as but not limited to a keyboard, a mouse, a joystick, a touchpad, a virtual keyboard, a virtual data entry user interface, a virtual dial pad, a software or a program, a scanner, a remote device, a microphone, a webcam, a camera, a fingerprint scanner, a cave, pointing stick.

The systems and methods can be practiced using any electronic device, which may be connected to one, or more of other electronic device with wires or wirelessly which may use technologies such as but not limited to, Bluetooth, Wi-Fi, WiMAX. This will also extend to use of the previously mentioned technologies to provide an authentication key or access key or electronic device based unique key or any combination thereof.

In some implementations, one or more users can be blocked or denied access to the smart suggester system.

Encryption can be accomplished using any encryption technology, such as the process of converting digital information into a new form using a key or a code or a program, where the new form is unintelligible or indecipherable to a user or a thief or a hacker or a spammer. The term ‘encryption’ includes encoding, compressing, or any other translating of the digital content. The encryption of the digital media content can be performed in accordance with any technology including utilizing an encryption algorithm. The encryption algorithm utilized is not hardware dependent and may change depending on the digital content. For example, a different algorithm may be utilized for different websites or programs. The term ‘encryption’ further includes one or more aspects of authentication, entitlement, data integrity, access control, confidentiality, segmentation, information control, and combinations thereof.

The described embodiments may be implemented as a system, method, apparatus or article of manufacture using standard programming and/or engineering techniques related to software, firmware, hardware, or any combination thereof. The described operations may be implemented as code maintained in a “non-transitory, computer readable medium”, where a processor may read and execute the code from the non-transitory, computer readable medium. A non-transitory, computer readable medium may comprise media such as magnetic storage medium (e.g., hard disk drives, floppy disks, tape, etc.), optical storage (CD-ROMs, DVDs, optical disks, etc.), volatile and non-volatile memory devices (e.g., EEPROMs, ROMs, PROMs, RAMs, DRAMs, SRAMs, Flash Memory, firmware, programmable logic, etc.), etc. The code implementing the described operations may further be implemented in hardware logic (e.g., an integrated circuit chip, Programmable Gate Array (PGA), Application Specific Integrated Circuit (ASIC), etc.).

Still further, the code implementing the described operations may be implemented in “transmission signals”, where transmission signals may propagate through space or through a transmission media, such as an optical fiber, copper wire, etc. The transmission signals in which the code or logic is encoded may further comprise a wireless signal, satellite transmission, radio waves, infrared signals, Bluetooth, etc. The transmission signals in which the code or logic is encoded is capable of being transmitted by a transmitting station and received by a receiving station, where the code or logic encoded in the transmission signal may be decoded and stored in hardware or a non-transitory, computer readable medium at the receiving and transmitting stations or devices. An “article of manufacture” comprises non-transitory, computer readable medium or hardware logic, and/or transmission signals in which code may be implemented. A device in which the code implementing the described embodiments of operations is encoded may comprise a non-transitory, computer readable medium or hardware logic. Of course, those skilled in the art will recognize that many modifications may be made to this configuration without departing from the scope of the present technology, and that the article of manufacture may comprise suitable information bearing medium known in the art.

The term network means a system allowing interaction between two or more electronic devices, and includes any form of inter/intra enterprise environment such as the world wide web, Local Area Network (LAN), Wide Area Network (WAN), Storage Area Network (SAN) or any form of Intranet.

The systems and methods can be practiced using any electronic device. An electronic device is selected from any device capable of processing or representing data to a user and providing access to a network or any system similar to the internet. The electronic device may be selected from but not limited to, personal computers, tablet computers, mobile phones, laptop computers, palmtops, portable media players, and personal digital assistants. In an embodiment, the computer readable medium data storage unit or data storage device is selected from a set of but not limited to USB flash drive (pen drive), memory card, optical data storage discs, hard disk drive, magnetic disk, magnetic tape data storage device, data server and molecular memory.

The process steps, method steps, algorithms or the like may be described in a sequential order, such processes, methods and algorithms may be configured to work in alternate orders. In other words, any sequence or order of steps that may be described does not necessarily indicate a requirement that the steps be performed in that order. The steps of processes described herein may be performed in any order practical. Further, some steps may be performed simultaneously, in parallel, or concurrently.

While the present invention is susceptible of embodiment in various forms, there is shown in the drawings and will hereinafter be described a presently preferred embodiment with the understanding that the present disclosure is to be considered an exemplification of the invention and is not intended to limit the invention to the specific embodiments illustrated. The use of “including”, “comprising” or “having” and variations thereof herein is meant to encompass the items listed thereafter and equivalents thereof as well as additional items.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude or rule out the presence or addition of one or more other features, integers, steps, operations, elements, components, and/or groups thereof.

While this detailed description has disclosed certain specific embodiments for illustrative purposes, various modifications will be apparent to those skilled in the art which do not constitute departures from the spirit and scope of the invention as defined in the following claims, and it is to be distinctly understood that the foregoing descriptive matter is to be interpreted merely as illustrative of the invention and not as a limitation.

1. A computer-implemented method performed by one or more processors, comprising:
receiving, by the one or more processors, data indicating a chief complaint relating to a patient during a doctor-patient interaction;
receiving, by the one or more processors, data indicating vitals relating to the patient;
receiving, by the one or more processors, data indicating symptoms relating to the patient;
generating, by the one or more processors, a treatment plan based on data indicating the chief complaint, data indicating the vitals, and data indicating the symptoms related to the patient; and
providing, by the one or more processors, the treatment plan to a client device of a doctor.

2. The computer-implemented method of claim 1, further comprising determining, by the one or more processors, a location of the doctor corresponding to the doctor-patient interaction.

3. The computer-implemented method of claim 1, further comprising determining, by the one or more processors, a location of the patient corresponding to the doctor-patient interaction.

4. The computer-implemented method of claim 2, further comprising determining, by the one or more processors, a profile for the doctor, wherein the profile for the doctor includes a type of specialty, doctor treatment plan preferences, and doctor clinical pathways preferences.

5. The computer-implemented method of claim 1, wherein the treatment plan comprises at least one of laboratories tests, tests, follow-ups, recommendations, allergies, and medications.

6. The computer-implemented method of claim 1, further comprising:
generating, by the one or more processors, weight factors based on the data indicating the chief complaint relating to the patient, the data indicating the vitals relating to the patient, and the data indicating the symptoms relating to the patient; and
assigning, by the one or more processors, the weight factors to patient data items to assist with generating the treatment plan.

7. The computer-implemented method of claim 5, wherein the patient data items comprise at least one of disease data items, treatment plan data items, treatment protocol data items, and clinical pathway data items.

8. A system comprising:
one or more computers and one or more storage devices storing instructions that are operable, when executed by the one or more computers, to cause the one or more computers to perform operations comprising:
receiving, by the one or more processors, data indicating a chief complaint relating to a patient during a doctor-patient interaction;
receiving, by the one or more processors, data indicating vitals relating to the patient;
receiving, by the one or more processors, data indicating symptoms relating to the patient;
generating, by the one or more processors, a treatment plan based on data indicating the chief complaint, data indicating the vitals, and data indicating the symptoms related to the patient; and
providing, by the one or more processors, the treatment plan to a client device of a doctor.

9. The system of claim 8, wherein operations further comprise determining, by the one or more processors, a location of the doctor corresponding to the doctor-patient interaction.

10. The system of claim 8, wherein operations further comprise determining, by the one or more processors, a location of the patient corresponding to the doctor-patient interaction.

11. The system of claim 9, wherein operations further comprise determining, by the one or more processors, a profile for the doctor, wherein the profile for the doctor includes a type of specialty, doctor treatment plan preferences, and doctor clinical pathways preferences.

12. The system of claim 8, wherein the treatment plan comprises at least one of laboratories tests, tests, follow-ups, recommendations, allergies, and medications.

13. The system of claim 8, wherein operations further comprise:
generating, by the one or more processors, weight factors based on the data indicating the chief complaint relating to the patient, the data indicating the vitals relating to the patient, and the data indicating the symptoms relating to the patient; and
assigning, by the one or more processors, the weight factors to patient data items to assist with generating the treatment plan.

14. The system of claim 8, wherein the patient data items comprise at least one of disease data items, treatment plan data items, treatment protocol data items, and clinical pathway data items.

15. A non-transitory computer-readable medium storing software comprising instructions executable by one or more computers which, upon such execution, cause the one or more computers to perform operations comprising:
receiving, by the one or more processors, data indicating a chief complaint relating to a patient during a doctor-patient interaction;
receiving, by the one or more processors, data indicating vitals relating to the patient;
receiving, by the one or more processors, data indicating symptoms relating to the patient;
generating, by the one or more processors, a treatment plan based on data indicating the chief complaint, data indicating the vitals, and data indicating the symptoms related to the patient; and
providing, by the one or more processors, the treatment plan to a client device of a doctor.

16. The computer-readable medium of claim 15, wherein operations further comprise determining, by the one or more processors, a location of the doctor corresponding to the doctor-patient interaction.

17. The computer-readable medium of claim 16, wherein operations further comprise determining, by the one or more processors, a location of the patient corresponding to the doctor-patient interaction.

18. The computer-readable medium of claim 17, wherein operations further comprise determining, by the one or more processors, a profile for the doctor, wherein the profile for the doctor includes a type of specialty, doctor treatment plan preferences, and doctor clinical pathways preferences.

19. The computer-readable medium of claim 18, wherein the treatment plan comprises at least one of laboratories tests, tests, follow-ups, recommendations, allergies, and medications.

20. The computer-readable medium of claim 15, wherein operations further comprise:
generating, by the one or more processors, weight factors based on the data indicating the chief complaint relating to the patient, the data indicating the vitals relating to the patient, and the data indicating the symptoms relating to the patient; and
assigning, by the one or more processors, the weight factors to patient data items to assist with generating the treatment plan.
